(19) United States
(12) Patent Application Publication
(10) Pub. No.: US 2021/0361567 A1
(43) Pub. Date: Nov. 25, 2021

(54) NICOTINE PARTICLES AND COMPOSITIONS

(71) Applicant: PHILIP MORRIS PRODUCTS S.A., Neuchatel (CH)

(72) Inventors: Gerard Zuber, Boulens (CH); Nicolo Volpe, Lausanne (CH)

(73) Assignee: PHILIP MORRIS PRODUCTS S.A., Neuchâtel (CH)

(21) Appl. No.: 17/394,623

(22) Filed: Aug. 5, 2021

Related U.S. Application Data
(62) Division of application No. 16/309,742, filed on Dec. 13, 2018.

(30) Foreign Application Priority Data
Jun. 30, 2016 (EP) 16177163.9

(51) Int. Cl. A61K 9/00; A61K 9/16; A61K 31/465; A61K 45/06

(52) U.S. Cl. CPC A61K 9/0075; A61K 9/1623; A61K 45/06; A61K 31/465; A61K 9/1617; A61K 9/1682

(57) ABSTRACT

The composition is formed of a plurality of particles. These particles include nicotine, a sugar, and an amino acid or short peptide.

This application is a divisional of U.S. patent application Ser. No. 16/309,742, filed 13 Dec. 2018, which is a § 371 U.S. National Stage of International Application No. PCT/IB2017/053543, filed 14 Jun. 2017, which claims the benefit of European Application No. 16177163.9, filed 30 Jun. 2016.

This disclosure relates to nicotine particles and compositions that are suitable for inhalation. These nicotine particles and compositions include nicotine, a sugar, and an amino acid.

Dry powder inhalers (DPI) are known and are used to treat respiratory diseases by delivering a dry powder comprising a pharmaceutically active compound, in aerosol form through inhalation to the patients' airways. In pharmaceutical dry powders, the active pharmaceutical ingredient (API) is usually agglomerated on the surface of larger carrier particles, such as lactose for example. DPI's operate complex mechanisms to ensure such agglomerates disperse, break up or disaggregate before the API is inhaled into the lungs.

It may be difficult to deliver nicotine particles to the lungs at inhalation at air flow rates that are within conventional smoking regime inhalation or air flow rates. Nicotine particles may have a tendency to agglomerate and stick to inhaler or processing surfaces, especially as a size of the nicotine particle deceases. Nicotine particles with an MMAD of less than about 10 micrometres tend to be increasingly thermodynamically unstable due to a high surface area to volume ratio, which provides an increasing surface free energy with this decreasing particle size, and consequently increases the tendency of particles to agglomerate and the strength of the agglomerate. Forming nicotine particles may be difficult and costly.

It would be desirable to provide nicotine particles and compositions that may be formed and processed easily. It would be desirable that the nicotine particles and compositions not stick to processing surfaces or agglomerate and exhibit a stable particle size distribution. It would also be desirable that the nicotine particles and compositions be deliverable to the lungs at air flow rates that are within conventional smoking regime inhalation or air flow rates.

This disclosure is directed to a particle that comprises nicotine, a sugar and an amino acid. The particle preferably has a size in a range from about 0.5 to about 10 micrometres, or from about 0.5 to about 5 micrometres. The particle preferably comprises about 25 wt % or less nicotine or from about 5 to about 15 wt % nicotine. A free flowing composition may be formed by these particles.

The particles may be formed by combining nicotine, a sugar, and an amino acid in a liquid carrier to form a liquid mixture. This liquid mixture is spray dried to form a plurality of particles having a size in a range from about 0.5 to about 10 micrometres or in a range from about 0.5 to about 5 micrometres. The plurality of particles is preferably homogenous particles.

Advantageously, the nicotine particles and powder formulation described herein provide for a homogenous and stable particle size sufficient to deliver nicotine to the lungs of a consumer at inhalation or air flow rates that are within conventional smoking regime inhalation or air flow rates. The nicotine particles and powder formulation described herein allows these particles to be formed by spray drying to achieve a specific and controlled particle size distribution while minimizing agglomeration or adherence to surfaces such as processing equipment surfaces. Spray drying may provide a scalable, precise and low cost particle formation unit operation.

The term “nicotine” refers to nicotine and nicotine derivatives in any form, including but not limited to, a free-base nicotine, nicotine salt, or in a matrix such as a sugar matrix or organometallic complex.

The term “amino acid” refers to a single unmodified or modified amino acid moiety, preferably unmodified.

The term “short peptide” refers to a peptide comprising two or three amino acids.

The size of a particle, as stated herein, preferably refers to the aerodynamic diameter of the particle. The aerodynamic diameter of a powder system is preferably measured with a cascade impactor. The term “MMAD” refers to the mass median aerodynamic diameter.

This disclosure relates to particles comprising nicotine, a sugar, and an amino acid.

Particles may be formed having a specific particle size distribution. In illustrative examples, about 90%, or about 95%, or about 98% of the particles have a size of about 5 micrometres or less, or about 4.5 micrometres or less, or about 4.2 micrometres or less, and about 50% of the particles have a size of about 2.5 micrometres or less, or about 2.1 micrometres or less. In many of these examples, about 10% of the particles have a size of about 820 nanometers or less. The particles may have a mass median aerodynamic diameter in a range from about 1 to about 4 micrometres. Substantially all of the particles may have a particle size in a range from about 500 nanometers to about 5 micrometres.

Compositions of these particles have a specific particle size distribution. In illustrative examples, about 90%, or about 95%, or about 98% of the particles of the composition have a size of about 5 micrometres or less, or about 4.5 micrometres or less, or about 4.2 micrometres or less, and about 50% of the particles have a size of about 2.5 micrometres or less, or about 2.1 micrometres or less. In many of these examples, about 10% of the particles have a size of about 820 nanometers or less. The particles of the composition may have a mass median aerodynamic diameter in a range from about 1 to about 4 micrometres. Substantially all of the particles forming the composition may have a particle size in a range from about 500 nanometers to about 5 micrometres. The percentages relating to particle size distribution described herein are based on particles by volume (% by volume).

The nicotine component of the particle may be a free base nicotine, a nicotine salt, or a combination thereof. The nicotine component may be a nicotine salt formed by combining nicotine or nicotine free base with an acid. The acid may be a stoichiometric amount of acid to the nicotine free base, or a stoichiometric excess of acid may be combined with the nicotine free base, or a stoichiometric excess of nicotine free base may be combined with the acid. A free base nicotine may be utilized without the addition of an acid.

The acid may be an organic acid, an inorganic acid, or a Lewis acid. Non-limiting examples of inorganic acids are hydrochloric, hydrobromic, hydroiodic, nitric, sulfuric, phosphoric, acetic, hexafluorophosphoric, and the like. Non-limiting examples of organic acids are levulinic, citric, gluconic, benzoic, propionic, butyric, sulfosalicylic, maleic, lauric, malic, fumaric, succinic, tartaric, amsonic, pamoic, mesylic, aspartic, formic, acetic, propionic, succinic, camphorsulfonic, fumaric, isethionic, lactic, mucic, para-toluenesulfonic, glycolic, glucuronic, maleic, furoic, glutamic, benzoic, anthranilic, salicylic, phenylacetic, pyruvic, mandelic, embonic (pamoic), methanesulfonic, ethanesulfonic, pantothenic, benzenesulfonic (besylate), stearic, sulfanilic, alginic, galacturonic, and the like. Non-limiting examples of Lewis acids are zinc chloride or zinc bromide (ZnCl₂/ZnBr₂). These can react with nicotine to form organometallic complexes.

Useful nicotine salts include, but are not limited to, nicotine pyruvate, nicotine citrate, nicotine aspartate, nicotine lactate, nicotine bitartrate, nicotine salicylate, nicotine fumarate, nicotine mono-pyruvate, nicotine glutamate or nicotine hydrochloride, for example. Preferred nicotine salts include, nicotine lactate, nicotine pyruvate, nicotine citrate, nicotine aspartate, or a combination thereof.

The pH of the particles (dissolved in water) may be in a range from about 5 to about 9. Preferably the pH is about 7.0 or higher or in a range from 7.0 to 9.0. A pH of 9 can be reached for a particle without organic acid, while a pH of 5.0 can be obtained with the use of a strong acid or diacid when forming the nicotine salt.

The particle may include an amino acid or peptide (preferably formed of three or less amino acids). The amino acid or peptide may reduce adhesion forces of the particles forming the composition and mitigate or prevent agglomeration of the particles forming the composition. The particles forming the composition described herein thus may be a free flowing material and possess a stable relative particle size distribution during processing, transport and storage. The amino acid may be a single amino acid or molecule containing two or more amino acids such as a peptide.

Useful amino acids may include leucine, alanine, valine, isoleucine, methionine, phenylalanine, tyrosine, tryptophan, or a combination thereof. One preferred amino acid is leucine or a leucine isomer such as, L-leucine. Useful peptides include trileucine, for example.

The particle may include a sugar. Sugar refers to simple sugars, monosaccharides, disaccharides, and polysaccharides. Without limitation, examples of suitable sugars are lactose, sucrose, raffinose, trehalose, fructose, dextrose, glucose, maltose, mannitol, or combinations thereof. Preferred sugars include trehalose or mannitol.

The particle may contain less than about 30 wt % nicotine. The particle may contain about 25 wt % or less nicotine, or from about 15 to about 25 wt % nicotine. The particle may contain from about 1 to about 20 wt % nicotine, or from about 10 to about 20 wt % nicotine, or from about 5 to 15 wt % nicotine. The particle may contain from about 1 to about 10 wt % nicotine or from about 5 to about 10 wt % nicotine. In some embodiments, particles that contained about 30 wt % or more nicotine agglomerated or adhered to processing surfaces when processed through a spray dryer.

The particles forming the composition may contain less than about 30 wt % nicotine. The particles forming the composition may contain about 25 wt % or less nicotine, or from about 15 to about 25 wt % nicotine. The particles forming the composition may contain from about 1 to about 20 wt % nicotine, or from about 10 to about 20 wt % nicotine, or from about 5 to 15 wt % nicotine. The particles forming the composition may contain from about 1 to about 10 wt % nicotine or from about 5 to about 10 wt % nicotine. In some embodiments, particles forming the composition that contained about 30 wt % or more nicotine produced an agglomerated or sticky composition when processed through a spray dryer.

The particle may contain about 1 to about 10 wt % amino acid. The particle may contain about 3 to about 7 wt % amino acid. The particle may contain from about 5 wt % amino acid. The addition of the amino acid, especially L-leucine for example, to the particles may reduce agglomeration or adherence to processing surfaces.

The particles forming the composition may contain about 1 to about 10 wt % amino acid. The particles forming the composition may contain about 3 to about 7 wt % amino acid. The particles forming the composition may contain from about 5 wt % amino acid. The addition of the amino acid, especially L-leucine for example, to the particles forming the composition may reduce agglomeration or stickiness of the composition when processed through a spray dryer.

The particle may contain about 60 to about 95 wt % sugar. The particle may contain about 70 to about 90 wt % sugar. The particle may contain about 80 to about 85 wt % sugar.

The particles forming the composition may contain about 60 to about 95 wt % sugar. The particles forming the composition may contain about 70 to about 90 wt % sugar. The particles forming the composition may contain about 80 to about 85 wt % sugar.

A useful particle formulation includes an amino acid being leucine, a sugar being trehalose, and a nicotine salt being nicotine lactate. The nicotine content may be from about 5 to about 15 wt % or about 9.5 wt %. The leucine content may be from about 1 to about 10 wt % The leucine content may be from about 3 to about 7 wt % or about 5 wt %. The molar ratio of acid:nicotine may about 1:1.

A useful particle formulation includes an amino acid being leucine, a sugar being trehalose, and a nicotine salt being nicotine citrate. The nicotine content may be from about 5 to about 15 wt % or about 9.6 wt %. The leucine content may be from about 1 to about 10 wt % The leucine content may be from about 3 to about 7 wt % or about 5 wt %. The molar ratio of acid:nicotine may about 0.25:1.

A useful particle formulation includes an amino acid being leucine, a sugar being trehalose, and a nicotine salt being nicotine pyruvate. The nicotine content may be from about 5 to about 15 wt % or about 9.8 wt %. The leucine content may be from about 1 to about 10 wt % The leucine content may be from about 3 to about 7 wt % or about 5 wt %. The molar ratio of acid:nicotine may about 0.6:1.

A useful particle formulation includes an amino acid being leucine, a sugar being trehalose, and a nicotine salt being nicotine aspartate. The nicotine content may be from about 5 to about 15 wt % or about 9.3 wt %. The leucine content may be from about 1 to about 10 wt % The leucine content may be from about 3 to about 7 wt % or about 5 wt %. The molar ratio of acid:nicotine may about 0.6:1.

The particles may be formed by: (1) combining a nicotine, a sugar, and an amino acid or peptide in a liquid carrier to form a liquid mixture; and (2) spray drying the liquid mixture to form particles having a size in a range from about 0.5 to about 10 micrometres or in a range from about 0.5 to about 5 micrometres.

An illustrative example comprises a preparation that includes a 20% nicotine free base and an acid (e.g., lactic, pyruvic or citric) combined in a liquid carrier. The molar ratio may be within the ranges 1.00:1.20 for nicotine:aspartic, pyruvic or lactic acid, and 0.33:0.50 for nicotine:citric acid. The liquid mixture may be incubated at about 30° C., for example, for about 1 to about 15 minutes, to allow the formation of a stable nicotine salt solution. A pharmaceutically acceptable sugar, (for example, trehalose or mannitol) and leucine may be added to form a liquid mixture. The liquid mixture may be spray dried by using a nozzle to atomize the liquid to form droplets, contacting the droplets with warm air, to dry and form dry particles, and collecting the particles. In this embodiment, after spray drying, 10% of the particles (by volume) may be below about 0.82 micrometre in size, 50% of the particles may be below about 2.1 micrometres in size and 90% of the particles may be below about 4.1 micrometres in size. The particles are substantially in the range of 0.5 to 4.2 micrometres.

The liquid carrier may be water, for example. The liquid mixture is flowable. The liquid mixture is configured to flow through an atomization or atomizer nozzle to form the precise or controlled particle size distribution. The particles or composition may be processed by spray drying to form a precise size distribution of particles. The particles and compositions described herein may tend to not agglomerate or stick to the surface of the spray drying equipment.

The particles and compositions described herein may be processed at a reduced temperature (as compared to conventional nicotine particle formation) resulting in a reduced product loss. For example, the particles and composition described herein may be spray dried at a temperature in a range from about 50 to 85 degrees Celsius.

A cough suppressant may be combined with the composition. Cough suppressants include, for example, menthol, camphor, turpentine oil (e.g., alpha-pinene, beta-pinene) and menthol derivatives (e.g., menthyl lactate, and menthyl salicylate).

The particles and compositions described herein may then be packaged for consumption. The particles and compositions described herein may be packaged in an inhalation delivery consumable element or contained within an inhalation delivery consumable element. An inhalation delivery consumable element may be a capsule, for example. The capsule may be by disposed in an inhalation device, such as a dry powder inhaler. The inhalation device may pierce the capsule and the fine particles may be entrained in the inhalation air for delivery to the lungs of a consumer.

The particles and compositions described herein and the inhalation delivery consumable element may be free of, or substantially free of carrier particles. The particles and compositions described herein and the inhalation delivery consumable element may be free of, or substantially free of particles that are greater than about 20 micrometres, or greater than about 50 micrometres, or greater than about 100 micrometres.

The nicotine may be dissolved in the liquid carrier to form the liquid mixture. The sugar may be dissolved in the liquid carrier to form the liquid mixture. The amino acid may be dissolved in the liquid carrier to form the liquid mixture. The liquid mixture may have about 20% w/v or less total solids, or about 15% w/v or less total solids, or a range of about 5 to 15% w/v total solids.

The nicotine particles described herein may be processed at a reduced (as compared to conventional nicotine particles) temperature that may result in reduced product loss. The spray drying inlet temperature and the outlet temperature may be reduced. The spray drying atomization pressure may be in a range from about 3 to about 7 bar, or 4 to about 6 bar, or about 5 bar.

The spray drying inlet temperature may be about 140 degrees Celsius or less, or about 135 degrees Celsius or less, or about 130 degrees Celsius or less, or in a range from about 100 to about 1500 degrees Celsius, or in a range from about 110 to about 140 degrees Celsius, or in a range from about 125 to about 135 degrees Celsius. The spray drying outlet temperature may be about 100 degrees Celsius or less, or about 95 degrees Celsius or less, or about 90 degrees Celsius or less, about 85 degrees Celsius or less, or about 80 degrees Celsius or less, or in a range from about 30 to about 90 degrees Celsius, or in a range from about 40 to about 90 degrees Celsius, or in a range from about 50 to about 85 degrees Celsius.

Specific examples are set forth in the tables below.

All scientific and technical terms used herein have meanings commonly used in the art unless otherwise specified. The definitions provided herein are to facilitate understanding of certain terms used frequently herein.

As used herein, the singular forms “a”, “an”, and “the” encompass embodiments having plural referents, unless the content clearly dictates otherwise.

As used herein, “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising,” and the like.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

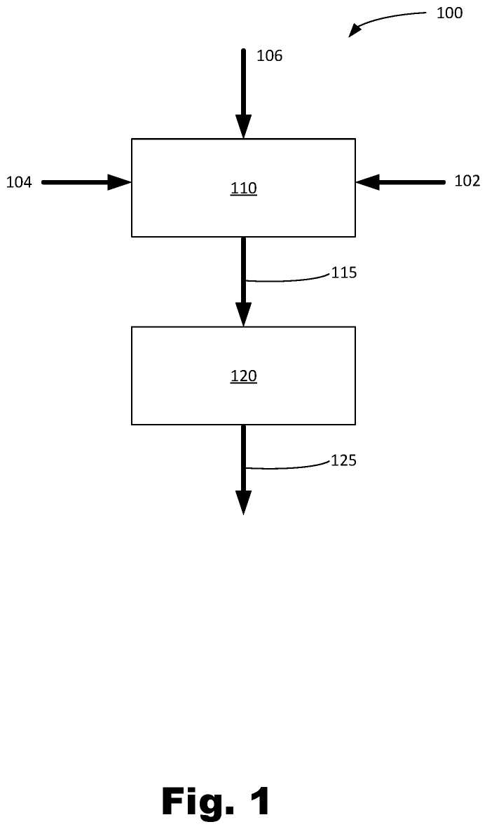

FIG. 1 is a schematic flow diagram of an illustrative method 100 of forming the particles 125. The method 100 includes combining nicotine 102, a sugar 104, and an amino acid or peptide 106 in a liquid carrier to form a liquid mixture 115 at block 110. Then, at block 120, the liquid mixture 115 is spray dried to form a plurality of particles 125.

### EXAMPLES

All the examples (except Table 3 examples) are formulated by combining a nicotine free base and an acid in water (at the specified ratio) to form a stable nicotine salt solution. Then the sugar and amino acid (leucine) is combined with the nicotine salt solution to form a liquid mixture. Then the liquid mixture is atomized and dried to form dry particles that are collected to from the composition.

The Table 3 examples are formulated by combining a nicotine free base with sugar and an amino acid (leucine) to form a liquid mixture. Then the liquid mixture is atomized and dried to form dry particles that are collected to from the composition.

The spray dryer was a Buchi B-290 spray dryer (available from Buchi Corp., DE, USA). The liquid mixture was provided to the spray dryer at a flow rate of 2 ml/min at 5 bar atomization pressure. The outlet temperature was about 80 degrees Celsius for examples utilizing trehalose. Table 1 below describes lactic acid formulations. Table 2 below describes pyruvic acid formulations. Table 3 below describes no acid formulations. Table 4 and Table 5 report the particle size distribution of various examples.

TABLE 1
Lactic Acid Nicotine Powder Formulations

| | | pH of | |
|---|---|---|---|
| | | powder | |
| Example | Formulation | solution | Comments |
| L1 | 10% Nicotine, Lactic acid (1:1), | 7.3 | Small amount of powder adhering |
| | 85% Trehalose | | to spray dryer surface |
| L2 | 15% Nicotine, Lactic acid (1:1), | 7.0 | Small amount of powder adhering |
| | 77% Trehalose | | to spray dryer surface |
| L3 | 10% Nicotine, Lactic acid (1:1), | 7.5 | Free flowing powder-no |
| | 80% Trehalose, 5% Leucine | | adherence |
| L4 | 15% Nicotine, Lactic acid (1:1), | 7.1 | Free flowing powder-no |
| | 72% Trehalose, 5% Leucine | | adherence |
| L5 | 20% Nicotine, Lactic acid (1:1), | — | Free flowing powder-no |
| | 64% Trehalose, 5% Leucine | | adherence |

TABLE 2
Pyruvic Acid Nicotine Powder Formulations

| | | pH of | |
|---|---|---|---|
| | | powder | |
| Example | Formulation | solution | Comments |
| P1 | 10% Nicotine, Pyruvic acid (0.6:1), | 7.5 | Powder adhering to spray dryer |
| | 87% Trehalose | | surface, cohesive powder |
| P2 | 15% Nicotine, Pyruvic acid (0.6:1), | 7.8 | Cohesive powder, some static |
| | 80% Trehalose | | charge |
| P3 | 10% Nicotine, Pyruvic acid (0.6:1), | 7.7 | Free flowing powder-no |
| | 82% Trehalose, 5% Leucine | | adherence, some static charge |
| P4 | 15% Nicotine, Pyruvic acid (0.6:1), | 7.8 | Free flowing powder-no |
| | 75% Trehalose, 5% Leucine | | adherence |
| P5 | 20% Nicotine, Pyruvic acid (0.6:1), | 7.7 | Free flowing powder-no |
| | 68% Trehalose, 5% Leucine | | adherence |

TABLE 3
No Acid (Free Base) Nicotine Powder Formulations

| | | pH of | |
|---|---|---|---|
| | | powder | |
| Example | Formulation | solution | Comments |
| N1 | 10% Nicotine, 90% Trehalose | 9.3 | Some powder adhering to spray |
| | | | dryer surface |
| N2 | 15% Nicotine, 85% Trehalose | 9.5 | Some powder adhering to spray |
| | | | dryer surface |
| N3 | 10% Nicotine, 85% Trehalose, 5% | 8.6 | Free flowing powder-no |
| | Leucine | | adherence, some static charge |
| N4 | 15% Nicotine, 80% Trehalose, 5% | 8.7 | Free flowing powder-no |
| | Leucine | | adherence |
| N5 | 20% Nicotine, 75% Trehalose, 5% | 8.8 | Free flowing powder-no |
| | Leucine | | adherence |

TABLE 4
Particle Size Distribution—reported in micrometres

| Example | X₁₀ | X₅₀ | X₉₀ | VMD |
|---|---|---|---|---|
| L1 | 0.65 | 1.43 | 3.54 | 1.81 |
| L2 | 0.68 | 1.62 | 3.75 | 1.97 |
| L3 | 0.76 | 1.89 | 3.86 | 2.14 |
| L4 | 0.92 | 2.14 | 3.99 | 2.35 |
| L5 | 0.78 | 1.95 | 3.90 | 2.19 |
| P1 | 0.67 | 1.54 | 3.47 | 1.85 |
| P2 | 0.67 | 1.53 | 3.54 | 1.86 |
| P3 | 0.66 | 1.48 | 3.54 | 1.84 |
| P4 | 0.72 | 1.78 | 3.79 | 2.06 |
| P4 | 0.65 | 1.43 | 3.54 | 1.81 |
| N1 | 0.68 | 1.62 | 3.75 | 1.97 |
| N2 | 0.76 | 1.89 | 3.86 | 2.14 |
| N3 | 0.92 | 2.14 | 3.99 | 2.35 |
| N4 | 0.78 | 1.95 | 3.90 | 2.19 |
| N5 | 0.67 | 1.54 | 3.47 | 1.85 |

X₁₀ refers to size of particle where 10% of particles, by volume, are less than this size.
X₅₀ refers to size of particle where 50% of particles, by volume, are less than this size.
X₉₀ refers to size of particle where 90% of particles, by volume, are less than this size.
VMD refers to volume mean diameter.

TABLE 5
Further Formulations

| Example | Formulation | X₁₀ | X₅₀ | X₉₀ | VMD | MMAD |
|---|---|---|---|---|---|---|
| 1 | 10% Nicotine, Lactic Acid (1:1), | 0.92 | 2.17 | 4.15 | 2.4 | 3.8 |
| | 80% Trehalose, 5% Leucine | | | | | |
| 2 | 10% Nicotine, Pyruvic Acid (1:0.6), | 1.04 | 2.56 | 5.08 | 2.9 | 4.0 |
| | 82% Trehalose, 5% Leucine | | | | | |
| 3 | 10% Nicotine, Citric Acid (1:0.25), | 0.81 | 2.34 | 5.48 | 2.8 | 3.5 |
| | 82% Trehalose, 5% Leucine | | | | | |
| 4 | 10% Nicotine, Aspartic Acid (1:0.6), | 0.82 | 2.24 | 4.96 | 2.6 | 4.2 |
| | 80% Trehalose, 5% Leucine | | | | | |
| 5 | 5% Nicotine, Lactic Acid (1:1), | 0.7 | 1.5 | 3.0 | 1.5 | 2.5 |
| | 82% Trehalose, 10% Leucine | | | | | |

1. A powder composition comprising a plurality of particles comprising nicotine particles, the nicotine particles comprising:
nicotine;
a sugar; and
an amino acid,
wherein about 90% of the plurality of particles have a particle size of about 4.5 micrometres or less, and about 50% of the plurality of particles have a particle size of less than about 2.5 micrometres.

2. The powder composition of claim 1, wherein the amino acid comprises leucine, alanine, valine, isoleucine, methionine, phenylalanine, tyrosine, or tryptophan.

3. The powder composition of claim 1, wherein the amino acid consists of leucine.

4. The powder composition of claim 1, wherein the nicotine comprises a nicotine salt.

5. The powder composition of claim 4, wherein the nicotine salt is a salt of nicotine base and levulinic acid, citric acid, gluconic acid, benzoic acid, propionic acid, butyric acid, sulfosalicylic acid, maleic acid, lauric acid, malic acid, fumaric acid, succinic acid, tartaric acid, amsonic acid, pamoic acid, mesylic acid, aspartic acid, formic acid, acetic acid, propionic acid, succinic acid, camphorsulfonic acid, fumaric acid, isethionic acid, lactic acid, mucic acid, para-toluenesulfonic acid, glycolic acid, glucuronic acid, furoic acid, glutamic acid, benzoic acid, anthranilic acid, salicylic acid, phenylacetic acid, pyruvic acid, mandelic acid, embonic (pamoic), methanesulfonic acid, ethanesulfonic acid, pantothenic acid, benzenesulfonic (besylate), stearic acid, sulfanilic acid, alginic acid, galacturonic acid, or a combination thereof.

6. The powder composition of claim 1, wherein the nicotine particles comprise from 5 wt % to 15 wt % nicotine.

7. The powder composition of claim 1, wherein the nicotine particles comprise from 60 wt % to 95 wt % sugar.

8. The powder composition of claim 1, wherein the nicotine particles comprise from 70 wt % to 90 wt % sugar.

9. The powder composition of claim 1, wherein about 10 vol % of the nicotine particles are below 0.82 μm in size.

10. The powder composition of claim 1, wherein about 50 vol % of the nicotine particles are below 2.1 μm in size.

11. The powder composition of claim 1, wherein about 90 vol % of the nicotine particles are below 4.1 μm in size.

12. The powder composition of claim 1, wherein the nicotine particles are substantially within a range of 0.5 μm to 4.2 μm in size.

13. The powder composition of claim 1, wherein the nicotine particles are prepared by spray drying a flowable liquid composition comprising the nicotine and a liquid carrier.

14. The powder composition of claim 13, wherein the nicotine particles are prepared at a temperature of 50° C. to 85° C.

15. The powder composition of claim 1 further comprising menthol.
